(19) United States
(12) Patent Application Publication
(10) Pub. No.: US 2026/0272625 A1
(43) Pub. Date: Sep. 17, 2026

(54) AN EXTRAORAL SCANNER SYSTEM

(71) Applicant: 3SHAPE A/S, Copenhagen K (DK)

(72) Inventors: Isak MOTTELSON, Copenhagen K (DK); Mike VAN DER POEL, Copenhagen K (DK)

(73) Assignee: 3SHAPE A/S, Copenhagen K (DK)

(21) Appl. No.: 19/167,991

(22) PCT Filed: Mar. 25, 2024
(86) PCT No.: PCT/EP2024/057951

§ 371 Date: Sep. 23, 2025

(30) Foreign Application Priority Data
Mar. 24, 2023 (DK) PA 2023 70149

(51) Int. Cl. A61C 9/00

(52) U.S. Cl. CPC A61C 9/006

(57) ABSTRACT

An extraoral scanner system for determining a 3D geometry of a dental object: at least one camera; a pattern generator configured to generate a probe light with a plurality of configurations in the form of an illumination pattern; an optical system configured to transmit the probe light towards the dental object, and to transmit at least a part of the light returned from the dental object to the camera to form a plurality of 2D dental data, an intraoral mirror device configured to reflect the probe light towards the dental object, and to transmit at least a part of the light returned from the dental object to the camera to form a plurality of 2D mirror dental data, and one or more processors.

### FIELD

The disclosure relates to an extraoral scanner system for determining a 3D geometry of at least a part of the surface of a dental object in an oral cavity. In particular, the extraoral scanner system is configured to determine 3D geometry information of at least a surface of a dental object via an intraoral mirror device.

### BACKGROUND

During a workflow of a dentist or a hygienist an 3D digital tooth impression of the dentition is performed with an intraoral scanner is a separate step from for example the regular visual inspection of teeth with an intraoral mirror device and explorer. The FOW (field of view) of typical the intraoral scanner makes it hard to scan an edentulous case, and it is also hard to achieve a high accuracy across a long distance as deviations from ground truth accumulates when small scan patches are stitched together to create a complete 3D model. In yet another example, the size of the tip can complicate data capturing in hard to reach areas like the backside of molars or event prevent intraoral scanning if the patient is not able to fully open the mouth. The intraoral scanner additionally requires a probe or tip to be inserted into the oral cavity. For hygienic reasons, this tip needs to be either covered with a (single use) microbial barrier or a sheath configured to cover the tip and be high-level disinfected in between patients. The scanner body may need to be covered with a (single use) microbial barrier or be intermediate level disinfected between patients to avoid cross contamination from the part in contact with the patient. These requirements translate into very demanding technical requirements on the time spend on scanning, design of the tip and the whole intraoral scanner system. It also necessitates a thorough cleaning and disinfection of the intraoral scanner by the operator between patients.

It is desirable to overcome the above-mentioned issues with an extraoral scanner system that includes an external scanner system and an intraoral mirror unit for capturing two-dimensional dental data.

### SUMMARY

An aspect of the present disclosure is to provide a method and a system that is able to perform an intraoral scanning while the dentist is performing an inspection of the teeth via an intraoral mirror device.

According to the aspects an extraoral scanner system for determining a 3D geometry of at least a part of the surface of a dental object in an oral cavity is disclosed. The system may include at least one camera accommodating an array of sensor elements; a pattern generator configured to generate, using a light source of the extraoral scanner system, and a probe light with a plurality of configurations in the form of an illumination pattern. The illumination pattern may be a static illumination pattern, such as a checkerboard pattern, or, the illumination pattern may be a coded time-varying illumination pattern or coded structured light, where a geometry or shape of the pattern is varying with time such as a temporal sequence of patterns used derive 3D geometry information. Intensity variation and/or color variation of pattern is not considered to be a time-varying illumination pattern. The light is provided in the form of the illumination pattern to provide a light oscillation on the object. The variation/oscillation in the pattern may be spatial, e.g. a static checkerboard or dot pattern, and/or it may be time varying. The system may include an optical system configured to transmit the probe light towards the dental object along a first optical path of the optical system thereby illuminating at least a part of the dental object with the illumination pattern, and to transmit at least a part of the light returned from the dental object to the at least one camera to form a plurality of 2D dental data. The system may further include an intraoral mirror device that is configured to reflect the probe light towards the dental object along a second optical path between the intraoral mirror device and the at least one camera, and to transmit at least a part of the light returned from the dental object via the second optical path to the at least one camera to form a plurality of 2D mirror dental data. The transmission of light via the first optical path does not involve the intraoral mirror device, which means, that the light is being reflected directly to the scanner without the need of involving the intraoral mirror device in redirecting the reflected light from the dental object such that the at least one camera captures the reflected light. The second optical path does involve the intraoral mirror device for transmitting the reflected light from the dental object to the at least one camera.

The intraoral mirror device may include a reflective surface that is configured to direct the reflected light from the dental object to the at least one camera. The system may include one or more processors configured to determine the 3D geometry of at least a part of the surface of the dental object based on the plurality of 2D dental data and the plurality of 2D mirror dental data and the illumination pattern.

The system may include a handheld dental scanner that includes the at least one camera, the pattern generator, the optical system and a tip configured to be inserted into the oral cavity. The handheld dental scanner may be an intraoral scanner device. The intraoral mirror device allows the handheld dental scanner to capture a plurality of 2D dental data, such as 2D images, of the oral cavity which the handheld dental scanner is not able to do because of the size of the tip of the scanner.

The system may include a headwear that includes the at least one camera, the pattern generator. The headwear may be a pair of glasses, a headband, a neckband, a hat or a cap. The at least one camera may be arranged such that when the user is able to visually see the dental object with at least one eye either directly, i.e. via the first optical path, or via the intraoral mirror device, i.e. via the second optical path, then the at least one camera is configured to capture the reflected light of a dental object. The reflective surface of the intraoral mirror device should be angled such that the user of the system is able to visually see the dental object of the oral cavity.

While the user of the system is performing an inspection of the teeth via the intraoral mirror device either when wearing the headwear or handling the intraoral scanner device, the one or more processors determines the 3D geometry of at least the dental object being inspected by the intraoral mirror device. Thereby, the systems allow the user to combine at least two working steps, such as inspection and intraoral scan, and thereby, reducing the amount of time the patient is needed to be seated in the clinic.

The one or more processors is configured to distinguish between light that is captured by the at least one camera via the first optical path or via the second optical path. The at least one camera may be configured to detect the polarization of the returned light, and the intraoral mirror device may include a polarizer, such as a Bragg mirror, that is configured to change the polarization of the reflected light from the dental object. Thereby, the light being captured by the at least one camera has different polarization depending on the optical path of the system. For example, the light that returns to the at least one camera via the first optical path has a first polarization, and the light that returns to the at least one camera via the second optical path has a second polarization. The first polarization is orthogonal to the second polarization. The one or more processors may be configured to identify the intraoral mirror device by detecting a polarization of the returned light, wherein the polarization of the returned light is different in the first optical path and in the second optical path.

The one or more processors may be configured to identify the intraoral mirror device via the at least one camera, and wherein the one or more processors may be further configured to identify the light returned via the intraoral mirror device based on the identification of the intraoral mirror device. Identification of the intraoral mirror device may be achieved by using a trained neural network to recognize and label the intraoral mirror in the plurality of 2D dental data.

The second optical path may be determined by the one or more processors by detecting via the at least one camera one or more identifiers of the intraoral mirror device. The second optical path may be determined by the one or more processors by detecting via the at least one camera a polarization of the light.

Ideally, the at least one camera may be used for 3D surface scanning as well as loupe function. The 3D surface scanning is used for determining the 3D geometry of at least a part of a surface of a dental object. The system may include a plurality of cameras arranged in stereo and won on a forehead by a headwear. The plurality of cameras is directed in such a way that they are aimed towards the patient's mouth when the dentist is sitting in a comfortable position to treat the patient and locking towards the mouth. The headwear may be glasses with screens that is configured to project stereo images captured by the plurality of cameras, and wherein the stereo images are mimicking the visibility of the eyes of the dentist. The cameras have macro-optics. A reasonable field of view (FOV) of the cameras is about 3 to 7 cm, about 4 cm or about 5 cm, and a working distance between the plurality of cameras and the mouth is about 20 cm to 40 cm, or about 30 cm to 40 cm. This FOV will allow to see about half of the dentition of the patient at any time. The light source may be arranged between the plurality of cameras. The light source would follow the head movement of the dentist, and thus, continuously illuminating the field of view. With parallel or near-parallel illumination between the line of sight of the eyes and the light source there will be little or no shadows in the illuminated scene.

The loupe function offers several advantages to the analogue: 1) It can provide digital zoom and digital image stabilization. This is difficult if not impossible to achieve in an analogue version of reasonable size. 2) It allows even more freedom to select a healthy posture of the dentist since the camera orientation and display device are not part of the same optical system. For instance, the cameras can be pointed more downwards than what is practically possible with analogue loupes. 3) It offers recording of images and videos that can be used for documentation, sharing during surgery, patient communication, etc.

The loupe function can in addition assist the dentist with information displayed in the glasses. The 2D stereo image can be registered onto a 3D model and allow for e.g. guided surgery, automatic pocket depth measurements, bracket placements, and much more.

It may be advantageous to use separate cameras with different properties for the 3D surface scanning and for the loupe function. The plurality of cameras may include a first camera for 3D surface scanning and a second camera for color imaging. The camera for the loupe function may be the second camera. The second camera may be a 4K cameras (4096×2160 pixels) that allows for higher resolution and more digital zoom. In the case of different set of cameras, it may be possible to use a beam splitter so the same macro-optics are serving the cameras. The system may include a set of cameras for the 3D surface scanning and a set of loupe cameras for the loupe function and color imaging, and wherein both sets have similar perspectives. The set of loupe cameras can be used for color imaging in high resolution and the obtained color texture can easily be applied to the 3D surface acquired by the set of 3D cameras. This may also allow to reduce the number of patterns needed for the 3D scanning so that the speed requirement of the 3D cameras is reduced for a given 3D frame rate.

Each of the cameras of the first set may include a monochrome sensor and a narrow bandpass filter for filtering the incoming reflected light. The center wavelength of the narrow bandpass filter should be similar to the wavelength of the probe light. This will reduce the effect of background light in the scanning situation. Ideally, the operator can keep the probe light on during the scanning since this will allow for the most seamless integration into the existing workflow. But if the bright dental light from the chair remains so strong that it reduces contrast needed for 3D scanning, then it may be necessary to turn off the dental light. Infront of a second camera, the second set of cameras for color imaging a filer that transmits the reflected light from above the wavelength of the probe light.

With separate cameras, it is also possible to enhance imaging of specular reflections from the probe light for the 3D scanning. This can be done in several ways. One will be to linearly polarize the probe light and apply a parallel linear filter in front of the array of sensor element of the camera used for 3D scanning. This will enhance contrast in the pattern image on the sensor since diffusely and thus depolarized light from the teeth will be partially suppressed. The contrast enhancement can make 3D scanning less noisy. At the same time, it is an option to suppress specular reflections in the sensor used for color imaging by application of a crossed polarizer configuration for this camera. This can give more truthful colors as the specular reflection is a highlight from the surface of e.g. teeth that do not sample the underlying tooth tissue and hence does not convey information on the tissue's color.

A probe light with narrow-band light puts less stringent requirements on the achromaticity of the system.

The one or more processors may be configured to identify a mirror surface plane of the intraoral mirror device by tracking the one or more identifiers via the at least one camera, and wherein the one or more processors may be configured to determine the second optical path based on the identified mirror surface plane. The mirror surface plane may be configured to reflect the probe light to the dental object and to reflect the returned light from the dental object to the at least one camera. The mirror surface plane may be configured to reflect the probe light and the returned light that include wavelength between 350 nm and 1100 nm. The mirror surface plane may be configured to reflect the probe light and the returned light with more than 50%. The mirror surface plane may include the polarizer, such as a Bragg mirror.

The one or more identifiers may include a geometry of the mirror surface plane, and the one or more processors may be configured to identify the geometry of the mirror surface plane by using a first neural network, and wherein the one or more processors may be configured to identify the light returned via the intraoral mirror device based on the geometry of the mirror surface plane. The first neural network may be trained based on a plurality of different geometry of intraoral mirror devices. The first neural network may be trained by inputting labeled information covering different type of intraoral mirror devices that includes different geometrical shape of the mirror surface plane and different sizes of the mirror surface plane. Thereby, the first neural network may receive as input, from the one or more processors, at least a part of the plurality of 2D dental data and provide as output data associated with the recognition of the dental mirror surface in the at least part of the plurality of 2D dental data.

A neural network used to recognize the dental mirror surface may be created that uses a deep learning framework such as TensorFlow or PyTorch. The model should be designed to take images as input and output a segmentation map.

This input data (ground truth) should include labeled images that contain different objects and their boundaries. Such images may be generated by using trained professionals to manually label the data or a pretrained machine learning solution for automatically labeling the training data.

The input data may subsequently be preprocessed such that each image is standardized and ready to be used as input for the neural network. This may include resizing, cropping, and normalizing the images. The training of the neural network model may be done using an appropriate loss function and optimization algorithm. The training process could involve adjusting the weights of the model so that it can accurately identify and segment different objects in the images (such as teeth, dental mirror, soft tissue, lips etc.). Once the model is trained an evaluation may be done to measure the performance. This can be done by comparing the segmentation maps produced by the model with the ground truth segmentation maps.

Alternatively, a deterministic algorithm may receive from the one or more processors the at least one identifier, and based on the identifier, the deterministic algorithm may be configured to output the at least geometry of the mirror surface plane and/or the size of the mirror surface plane to the one or more processors. Based on the output, the one or more processors may be configured to detect the intraoral mirror device or the mirror surface plane in the 2D image that is captured by the at least one camera based on the returned light.

The first optical path is known through a calibration of the extraoral scanner system, however, the second optical path is changing while scanning as the relative position between the intraoral mirror device and the dental object is changing due to movement of the dental object and/or of the intraoral mirror device. It would then be of an advantage that the one or more processors may be configured to determine the second optical path in real time. Thereby, the quality of the 3D geometry is improved as error in the stitching of the plurality of 2D dental data and the plurality of 2D mirror dental data is reduced significantly.

At least the part of surface of the dental object may include a dental object plane, and each of the plurality of 2D dental data includes surface information of the dental object that corresponds to the dental object plane, and each of the plurality of 2D mirror dental data includes mirror surface information of the dental object that corresponds to the mirror surface plane, and wherein the one or more processors may be configured to transform the mirror surface information from the mirror surface plane to the dental object plane by a scaling function. Thereby, the one or more processors may be configured to stitch the plurality of 2D dental data together with the transformed plurality of 2D mirror dental data to determine the 3D geometry of at least the part of the surface of the dental object. In this example the 3D geometry of at least the part of the surface includes both a facial part and a lingual part. Without doing the transformation of the plurality of 2D mirror dental data, the one or more processors may be configured to determine the 3D geometry of at least the part of the surface, wherein the 3D geometry may be visual seen as two separate 3D geometries that includes a first 3D geometry of a facial part of the dental object and a second 3D geometry of a lingual part of the dental object.

The one or more processors may be configured to determine the scaling function, and wherein the scaling function is determined based on coordinates of the mirror surface plane relative to the at least one camera. The one or more processors may be configured to determine the scaling function, and wherein the scaling function may be determined by a mirror surface angle of the mirror surface plane relative to the at least one camera and a distance between the mirror surface plane and the at least one camera.

The determination of the 3D geometry of the surface of the dental object may be carried out by the one or more processors. The one or more processors may be configured to determine a first point cloud by performing triangulation of each of the plurality of 2D dental data, and wherein the one or more processors may be configured to determine a second point cloud by performing triangulation of each of the plurality of 2D mirror dental data, and the one or more processors may be configured to determine the 3D geometry by combining the first point cloud and the second point cloud. The 3D geometry may be determined in real time.

The illumination pattern may be a static pattern that includes-dark and bright regions, or, the illumination pattern may a time-varying illumination pattern, or, the illumination pattern may be a pseudo-random pattern.

Another central element of the disclosure is the probe light with an illumination pattern that is projected on to the object being scanned. The pattern may be static or time varying. The time varying pattern may provide a variation of light and darkness on and/or in the object. Specifically, when the pattern is varied in time then the in-focus regions on the object will display an oscillating pattern of light and darkness. The out-of-focus regions will display smaller or no contrast in the light oscillations. The static pattern may provide a spatial variation of light and darkness on and/or in the object. Specifically, the in-focus regions will display an oscillating pattern of light and darkness in space. The out-of-focus regions will display smaller or no contrast in the spatial light oscillations.

Light may be provided from an external light source, however preferably the scanner system comprises at least one light source and pattern generation means to produce the pattern. It is advantageous in terms of signal-to-noise ratio to design a light source such that the intensity in the non-masked parts of the pattern is as close to uniform in space as possible. In another embodiment the light source and the pattern generation means is integrated in a single component, such as a segmented LED. A segmented LED may provide a static pattern and/or it may provide a time varying pattern in itself by turning on and off the different segments in sequence. In one embodiment of the invention the time varying pattern is periodically varying in time. Such a time-varying pattern may be made with a DLP projector or multiple LEDs with different patterns that blinks in succession. In another embodiment of the invention the static pattern is periodically varying in space.

Light from the light source may be transmitted through the pattern generation means thereby generating the pattern. For example, the pattern generation means comprises at least one translucent and/or transparent pattern element. For generating a static pattern a opaque glass plate with an thinfilm metal mask can be used. E.g. the mask comprises a line pattern or checkerboard pattern. In general said mask preferably possesses rotational and/or translational periodicity. The pattern element is located in the optical path. Thus, light from the light source may be transmitted through the pattern element, e.g. transmitted transversely through the pattern element. The time varying pattern can then be generated by rotating, translating the pattern element or changing the pattern element via a mems mirror. A pattern element generating a static pattern does not need to be moved during a scan.

The system may comprise a handheld intraoral scanner that includes the at least one camera, the pattern generator, the optical system and a tip configured to be inserted into the oral cavity.

The system may comprise a headwear that includes the at least one camera, the pattern generator, and the optical system.

The handheld intraoral scanner or the headwear may include a wireless interface configured to transmit the plurality of 2D dental data and the plurality of 2D mirror dental data or the 3D geometry of at least the part of the surface of the dental object. The handheld intraoral scanner or the headwear may include a battery unit that comprises a rechargeable battery. The system may comprise a power management unit that is configured to optimize the power consumption of the headwear or the handheld intraoral scanner. The power management unit may be configured to set the extraoral scanner system into different power modes based on an input from the at least one camera, wherein a dental object is detected by the power management unit or the processing unit via the at least one camera, and the power management unit may be configured to set the extraoral scanner system into either the scan mode, the loop mode, the colour imaging mode, and/or the infrared imaging mode. For example, the at least one camera detects a loop device, then the power management unit is configured to set the extraoral scanner system into the loop mode. Furthermore, the at least one camera detects the intraoral mirror device, then the power management unit is configured to set the extraoral scanner system into the scan mode, the colour imaging mode and/or the infrared imaging mode. The power management unit may be configured to set the extraoral scanner system into different power modes based on an input from the at least one camera. In one example, the at least one camera does not detect a dental object or the intraoral mirror device, the power management unit or the processing sets the system into a standby mode during a period of time

The headwear includes a movement detection unit that includes at least a gyroscope and/or an accelerometer configured to detect a movement of the headwear. The power management unit may be configured to set the extraoral scanner system into different power modes based on an input from the movement detection unit, wherein the power management unit may be configured to set the extraoral scanner system into a different power mode than a standby mode if a movement of the headwear is detected by the movement detection unit during a period of time. In an example, where the headwear has not been used for few minutes or seconds, then the power management unit sets the system into the standby mode, which may imply turning of the light source or turning down the power of the light source, turning off the at least one camera and/or part of other units of the system.

The power management unit may be configured to set the extraoral scanner system into different power modes based on an input from a user interface, wherein the user interface may be arranged on the headwear, and the user interface may include a button. For example, when a user of the system interacts with the button then the power management unit turns the system into a power mode that is different from the standby mode.

To determine real time 3D geometry, the camera may be a high-speed camera. The high-speed camera may have a continuous frame rate of above 200 2D images per second, or above 500 2D images per second. The system may comprise a plurality of high-speed cameras that includes the at least one camera, and wherein the plurality of high-speed cameras is configured to generate the plurality of 2D dental data and the plurality of 2D mirror dental data in real time.

To minimize blur effect in the capture 2D images, i.e. the plurality of 2D dental data, it would be advantageous to have a non-continuous frame rate per second that includes multiple burst scans that includes a burst frame rate of about 10 to 30 2D images per 0.1 second, and the non-continuous frame rate per second would still be above 200 2D images per second, or above 500 2D images per second. That means between each of the multiple burst scans a short break will appear such that the non-continuous frame rate per second is maintained at above 200 2D images per second or above 500 2D images per second. By applying these burst scans would minimize the blur effect in the captured 2D images. The plurality of highspeed cameras may include 2 or 4 individual highspeed cameras. The pattern generator, the light source and the optical system may be configured to project a structured light pattern on the teeth from the line of sight, and the plurality of cameras are arranged such that the projected pattern is observed from an angle relative to the light source. In the example where the headwear includes the plurality of highspeed cameras, the pattern generator, and the optical system, the plurality of highspeed cameras may be directed in such a way that they are aimed towards the patient's mouth when the dentist is sitting in a comfortable position to inspect the patient with the intraoral mirror device. A reasonable Field-Of-View (FOV) of the plurality of highspeed cameras may be about 3 to 10 cm and working distance may be about 30 to 60 cm. The FOV will allow to see about half of the dentition of the patient at any time. The light source may be arranged between the two cameras and which will follow the head movement and thus continuously illuminate the observed field by the plurality of highspeed cameras. With parallel or near-parallel illumination between the light pattern and the FOV there will be little or no shadows in the illuminated scene.

The plurality of high-speed cameras has a camera field-of-view axis, and the pattern generator has a light field-of-view-axis, and the plurality of high speed camera is arranged according to the light source such that an angle for each of the plurality of high-speed camera between the camera field-of-view axis and the light field-of-view-axis may be between 4° and 200, 6° and 10°, about 8° or about 10°.

The plurality of cameras or the at least one camera may be a plenoptic camera, i.e. a light-field camera with the capability of capturing a plurality of images.

The intraoral mirror device may include the pattern generator, and wherein the one or more processors may be configured to perform real time calibration of the plurality of 3D dental data by monitoring the multiple identifiers on the intraoral mirror device. By applying the light source on the intraoral mirror device, it is avoided uncomfortable light being shined into the eyes of the patient. The intraoral mirror device may include a button interface for turning on and off the light source.

The one or more processors may be configured to determine corresponding features across the plurality of 2D dental data and the plurality of 2D mirror dental data by solving a correspondence problem algorithm and perform triangulation of the corresponding features to obtain 3D surface information from the plurality of 2D dental data and the plurality of 2D mirror dental data, and the one or more processors may be configured to stich the plurality of 3D surface information to form the 3D geometry of the dental object.

The highspeed camera may comprise multiple A/D converters per line of pixels, e.g. at least 2, 4, 8 or 16 A/D converters per line of pixels.

The plurality of high-speed camera may be arranged symmetrically or asymmetrically according to the light source. The symmetrically arrangement of the plurality of highspeed cameras is ideal for a stereo configuration of the plurality of high speed cameras. With the asymmetrical arrangement of the plurality of highspeed cameras, the one or more processors is configured to determine the 3D geometry based on two triangulation angles. One angle for determining a first depth, and another angle for determining a second depth, wherein the first depth is less than the second depth, or vice versa. The two depth 2D dental data may be combined to form the 3D geometry.

The probe light may include one or more wavelengths between 380 nm and 425 nm. In this example, the ratio between the probe light and the ambient light has increased significantly without the need for increasing the power of the probe light. It is known that the power of the ambient light between 380 nm and 425 nm is way lower than above 425 nm. Thereby, the system becomes more insensitive to ambient light, and the Signal-to-noise ratio of the captured light is improved significantly.

The system may include two or more light sources, wherein the two or more light sources includes the light source configured to emit light at a first wavelength and at least another light source configured to emit light at a second wavelength, and wherein the system may be configured to switch between the light source and the at least another light source. The switching between the two light sources may be between [200 and 400] ms or within [0-200] ms. Thereby, the one or more processors may be configured to capture reflected light via the at least one camera with different modalities, and in a way, that no substantial relative movement between the dental object in between capturing data in the two different modalities, such as between the modality and the second modality. The first modality may include one or more visible wavelengths and the second modality may include one or more near-infrared wavelengths.

The system may be configured to be in a mode of operation such as, a scan mode for acquiring the plurality of 2D dental data, such as intraoral 2D dental data, a loupe mode for acquiring still images or video images at different zoom settings of the at least one camera, a color imaging mode for acquiring color imaging with a higher resolution compared to the scan mode, and/or an infrared imaging mode for acquiring images of within the at least dental object. Furthermore, the system may be configured to operate in a dual mode that may include at least two of the following modes; scan mode, the loupe mode, the color imaging mode, and the infrared imaging mode. Furthermore, the system may be configured to shift between the modes during a mode of use of the system. Thereby, the system becomes flexible in use.

If the at least one camera is arranged in the head, then the system may include an automatic focusing of the at least one camera. The system may include a controller that includes an autofocusing function configured to control a position of at least an optical lens of an optical lens system of the at least one camera based on an input from the movement unit, and based on the position of at least the optical lens will change the focusing plane of the at least one camera.

While performing the inspection and scanning, the system is configured to provide visual feedback of the plurality of 2D dental data and/or the plurality of 2D mirror dental date. The visual feedback may include 2D information and/or the 3D geometry, i.e. a 3D model determined based on the 3D geometry, in real time. That means the user is able to see in real time the inspection images that includes the plurality of 2D mirror dental date and/or the 3D model that includes both the plurality of 2D mirror dental date and the plurality of 2D dental data, on a displaying unit.

The displaying unit may be arranged external to the headwear or part of the headwear, wherein the displaying unit may be configured to display one or more of the plurality of 2D dental data and/or one or more of the plurality of 2D mirror dental data.

The displaying unit may be configured to display solely or a combination of still images, video images at different zoom settings, color imaging, infrared imaging mode.

The displaying unit may be part of the headwear, the system comprises another displaying unit configured to display the same as the displaying unit.

For improving the inspection with the intraoral mirror device and the scanning of the oral cavity, the patient may have a mouth piece inserted into his/hers mouth to keep the mouth open and in a fixed relative position between the jaws. The mouthpiece may be made of a transparent material in relation to the emitted light of the projector unit, wherein the mouthpiece may configured to maintain an open mouth that includes the at least dental object.

The system may include a guiding mean unit configured to determine guiding instructions to a displaying unit or to a speaker unit part of the system or a headwear, wherein the guiding instructions includes guidance on how to position a user's head wearing the headwear in order to acquire optimal intraoral scan data, still images, video images, color images and/or infrared images.

The headwear may be a pair of glasses, a headband, a neckband, a hat, or a cap.

The light source may be configured to emit light within a range of 400 nm to 2000 nm.

The at least one camera and/or the at least second camera may include at least a first image sensor and an optical lens system for collimating the incident beam to the at least first image sensor. A further image sensor may be includes, wherein the optical lens system may ne configured to be shared between the first image sensor and the second image sensor. Furthermore, a dichroic mirror may be arranged between the optical lens system and the first image sensor and between the optical lens system and the second image sensor. The dichroic mirror is configured to reflect the incident beam within a first group of wavelengths to the second image sensor, and the dichroic mirror is further configured to transmit the incident beam with a second group of wavelengths to the first image sensor. The first group of wavelengths may be visible wavelengths and the second group of wavelengths may be invisible wavelengths, such as infrared or near infrared.

The user may look into the eyes of the patient to converse with the patient but the emitted light from the light source would be uncomfortable for the patient. In this case there is a need for reducing the power or turning off the light source.

The one or more processors may be configured to reduce the power or turn off the emitted light source when the at least one camera captures 2D images that cannot be stitched to the 3D model.

The one or more processors may be configured to reduce the power or turn off the emitted light source when the at least one camera captures an eye of a patient. The system may include a memory that includes eye reference data which the one or more processors are using for identifying an eye in the captured 2D images. The identification of an eye is provided by aligning eye reference data to different parts of the 2D images, and when a successful alignment is obtained, the light source is turned off or turned down in power.

When the light is turned off the user needs to turn on the light source manually via a user interface/graphical user interface in the system.

When the light source is turned down in power the at least one camera is still able to capture 2D images of the dental object in the oral cavity of the patient. When the at least one camera captures a dental object or an oral cavity, the power of the light source is turned up to an optimal scanning power. In this example, the one or more processors is configured to turn up the power of the light source either when the captured 2D images include 2D dental data that can be stitched together with the 3D model or when the captured 2D images include an oral cavity.

The extraoral scanner system may be with a limited 3D scanning quality which can be used for recording differences to an earlier high-quality baseline scan determined by an intraoral scanner with optimal 3D scanning quality. The differences may be teeth movement or changes in caries lesions. For a patient that does not have significant development in the oral status, a baseline scan will probably be applicable for many years. The system can identify if there are so large changes in the oral scene that the extraoral scanner system cannot track, and hence a new baseline scan is needed.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an example of an extraoral scanner system;

FIGS. 2A to 2G illustrate different examples of the extraoral scanner system;

FIG. 3 illustrates an example of where a first dental object and a second dental object is captured by at least one camera or multiple cameras;

FIGS. 4A and 4B illustrate different configurations of an arrangement including a pattern generator, an optical system and at least one camera;

FIGS. 5A, 5B and 5C illustrate different examples of a camera;

FIGS. 6A and 6B illustrate different examples of how to identify an intraoral mirror device;

FIG. 7 illustrates an example of the one or more processors configured to transform the captured mirror dental data that are depicted on an intraoral mirror device;

FIG. 8 illustrates another example on how to determine whether the reflected light captured by at least one camera; and

FIG. 9 illustrates an example of an extraoral scanner system.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intra-oral scan data may be performed by a dental scanning system that may include an intraoral scanning device such as the TRIOS series scanners from 3Shape A/S or a headwear. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is capable of obtaining surface information by operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The extraoral scanning device is generally moved and angled relative to the dentition while the user is moving, for example, his head during an inspection session with an intraoral mirror device. In this example, the inspection session becomes also a scanning session, where the user is able to inspect teeth via the intraoral mirror device while the extraoral scanning device acquires 2D images directly and indirectly via the intraoral mirror device of the teeth. At least some sets of sub-scans overlap at least partially, in order to enable reconstruction of the digital dental 3D model by stitching overlapping subscans together in real-time and display the progress of the virtual 3D model on a display as a feedback to the user. The result of stitching is the digital 3D representation of a surface larger than that which can be captured by a single sub-scan, i.e. which is larger than the field of view of the 3D scanning device. Stitching, also known as registration and fusion, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Stitching errors may occur when attempting to merge multiple 3D scan images together. these errors may occur when the 3D model is not properly aligned with previous 3D scan images and may resulting in gaps, overlap, or misalignment in the 3D model. Stitching errors may additionally cause artifacts in the model, such as incorrect edges between objects or incorrect surface normal resulting in an inaccurate 3D model.

In one example, 3D reconstruction may process 3D scan images or subscans one by one as they arrive from the scanner. Each subscan may be aligned to the global model and its alignment (a rigid transformation matrix) may then be fixed for the rest of the reconstruction, until scanning is stopped. In particular, a subscan entering the reconstruction pipeline may not influence the already established alignment of previous subscans. Each subscan alignment has some amount of error, and the error also propagates to the alignment error for subsequent subscans. This can accumulate to very large global alignment errors over the course of scanning, especially if the scanner is moved in certain paths where loops occur. This can manifest itself as so-called “split teeth”, where different sides of the same tooth are displaced by up to millimeter. So, the current solution can in some cases produce an inaccurate 3D model.

An inaccurate 3D model may cause a wide range of problems as new 3D scan images can be correctly placed on the model or restorations designed on an in accurate model may not fit the patient upon manufacturing. The extraoral scanning device may have a relatively large FOW from the direct line of sight from the cameras. This may result in a 3D scan image containing a wide portion of the dental arch as view from the facial/labial side. By increasing the 3D scan image area fewer scans are needed to cover the dental arch which thereby reduces accumulated stitching errors. Even though, 3D scan data from the lingual side of the dental arch may be acquired partially by use of dental mirror with a smaller FOW, the model framework for 3D reconstruction may use the 3D scan data from the direct line of sight as origin for adding the lingual data obtained through the mirror. This provides a highly accurate model where stitching errors originating from the 3D scan data obtained via the mirror is reduced by constraining the stitching according to the labial/facial 3D scan data obtained via the large FOW from direct line of sight. the digital 3D representation of the surface includes stitched 3D images that have been acquired directly and indirectly via the intraoral mirror device of the teeth.

Another example of a scanning device is a triangulation scanner, where a time varying pattern is projected onto the dental object and a sequence of images of the different pattern configurations are acquired by one or more cameras located at an angle relative to the projector unit. In some embodiments, the intraoral scanner is based on triangulation, wherein at least one camera and a projector unit are positioned such that they form a triangle with respect to a point on the scanned surface. As an example, a projector and a camera may be utilized to determine points in 3D space based on triangulation. Alternatively, the intraoral scanner may comprise two or more cameras viewing the scene or scanned object from two different directions, wherein the cameras are configured to acquire a set of images, wherein a correspondence problem is solved based on triangulation. The correspondence problem generally refers to the problem of ascertaining which parts of one image correspond to which parts of another image. Specifically, the projector unit may be configured to project a plurality of projector rays, which are projected onto a surface of the dental object. In particular, solving the correspondence problem may include the steps of determining image features in the images within a set of images, and further associate said image features with a specific projector ray. Subsequently, the depth of each projector ray may be computed, whereby a 3D representation of the scanned object may be generated.

The scanning device may be a handheld intraoral scanner that is configured to be handled by the hand of a user during scanning of a patient. The ergonomic design of the scanning device, i.e. a housing of the scanner, is designed such that a single hand is able to hold the scanner during a scanning session. The scanning device may be a headwear that is configured to be worn on a head of a user during scanning or inspection of a patient. The design of scanning device, i.e. the headwear, is designed such that no interaction with the user's hands are needed except of handling an intraoral mirror device.

Color texture of the dental object may be acquired by illuminating the object using different monochromatic colors such as individual red, green and blue colors or my illuminating the object using multichromatic light such as white light. A 2D image may be acquired during a flash of white light.

Generally the process of obtaining surface information in real time of a dental object to be scanned requires the scanning device to illuminate the surface and acquire high number of 2D images. Typically a high speed camera is used with a framerate of 200-2000 2D frames pr second dependent on the technology and 2D image resolution. The high amount of image data needed to be handled by the scanning device to eighter directly forward the raw image data stream to an external processing device or performing some image processing before transmitting the data to an external device or display. This process requires that multiple electronic components inside the scanner is operating with a high workload thus requiring a high demand of current.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental scanning system preferably further comprises a processor configured to generate scan data (such as extra-oral scan data and/or intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

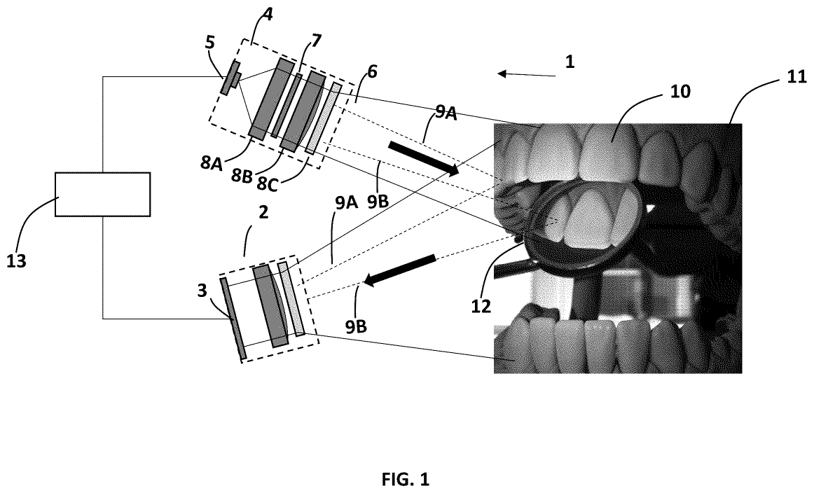

FIG. 1 illustrates an example of an extraoral scanner system 1 that is configured to determine a 3D geometry of at least a part of a surface of a dental object 10 in an oral cavity 11. The system 1 includes at least one camera 2 accommodating an array of sensor elements 3; a pattern generator 4 configured to generate, using a light source 5, a probe light 6 with a plurality of configurations in the form of an illumination pattern. In this example, the illumination pattern may be formed by a checkerboard 7, or a time-varying pattern generator 7 that is configured to varying in time the geometry or shape of the illumination pattern 6. Furthermore, the system 1 includes an optical system (8A,8B,8C), which in this example is arranged within the pattern generator 4, but in other examples, it could be arranged external to the pattern generator 4 together with the checkerboard 7or the time-varying pattern generator 7. In this example, the optical system (8A,8B,8C) includes at least two lenses (8A,8B) to collimate the light from the light source 5 and to collimate the probe light. The optical system does also include a wave plate polarizer 8C. The purpose of the optical system (8A,8B,8C) is to transmit the probe light towards the dental object 10 along a first optical path 9A of the optical system for illuminating at least a part of the dental object 10 with the illumination pattern, and to transmit at least a part of the light returned from the dental object 10 to the at least one camera 2 to form a plurality of 2D dental data. Furthermore, the system 1 includes an intraoral mirror device 12 configured to reflect the probe light 6 towards the dental object along a second optical path 9B between the intraoral mirror device 12 and the at least one camera 2, and to transmit at least a part of the light returned from the dental object 10 via the second optical path 9B to the at least one camera 2 to form a plurality of 2D mirror dental data. The system 1 further includes one or more processors 13 configured to determine the 3D geometry of at least a part of the surface of the dental object 10 based on the plurality of 2D dental data and the plurality of 2D mirror dental data and the illumination pattern.

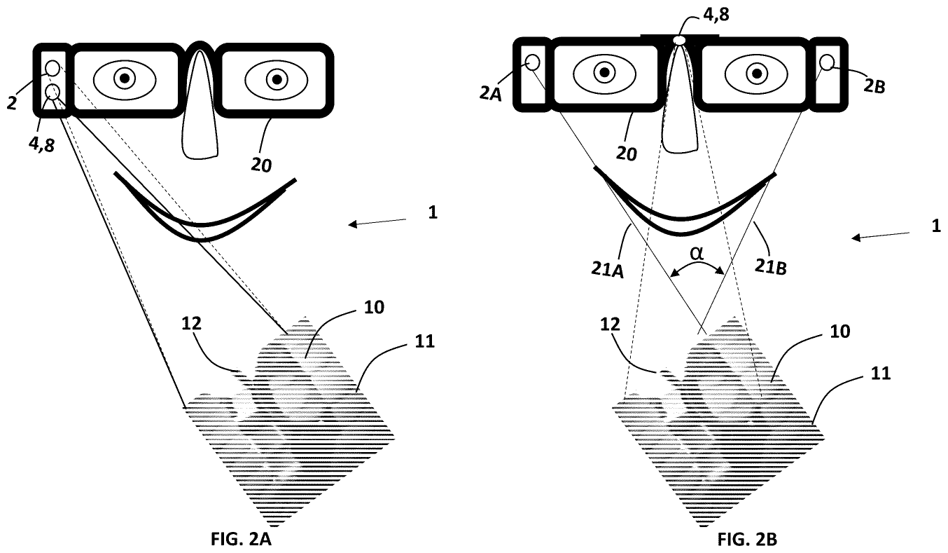

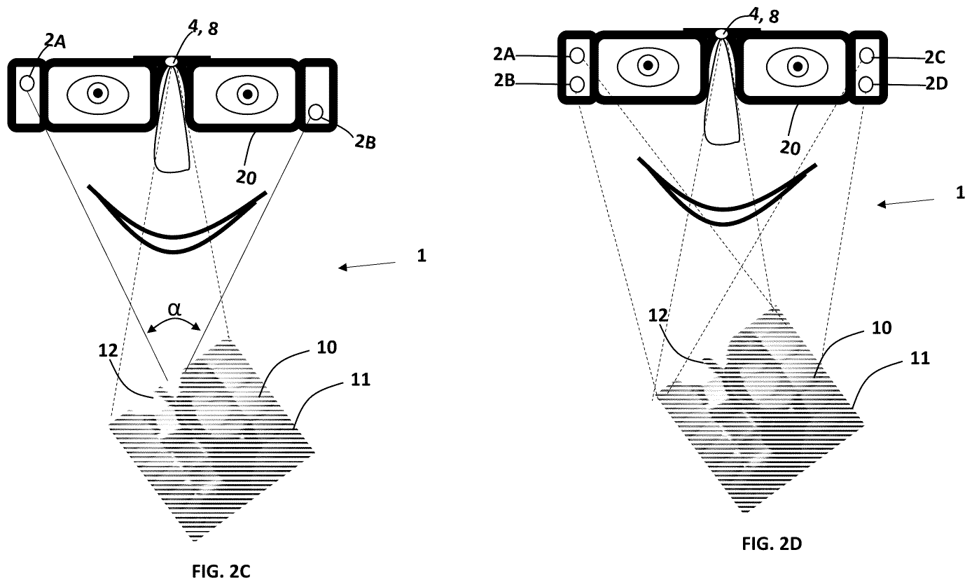

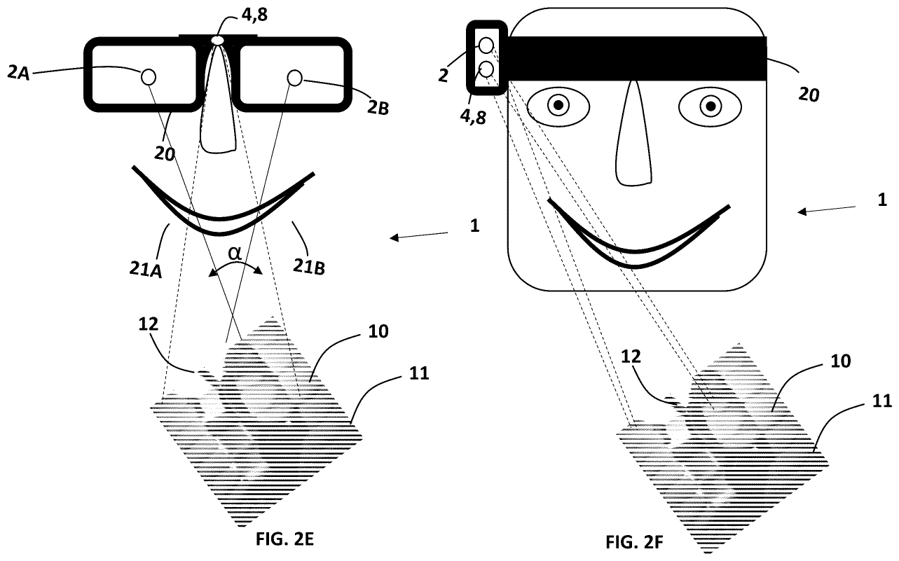

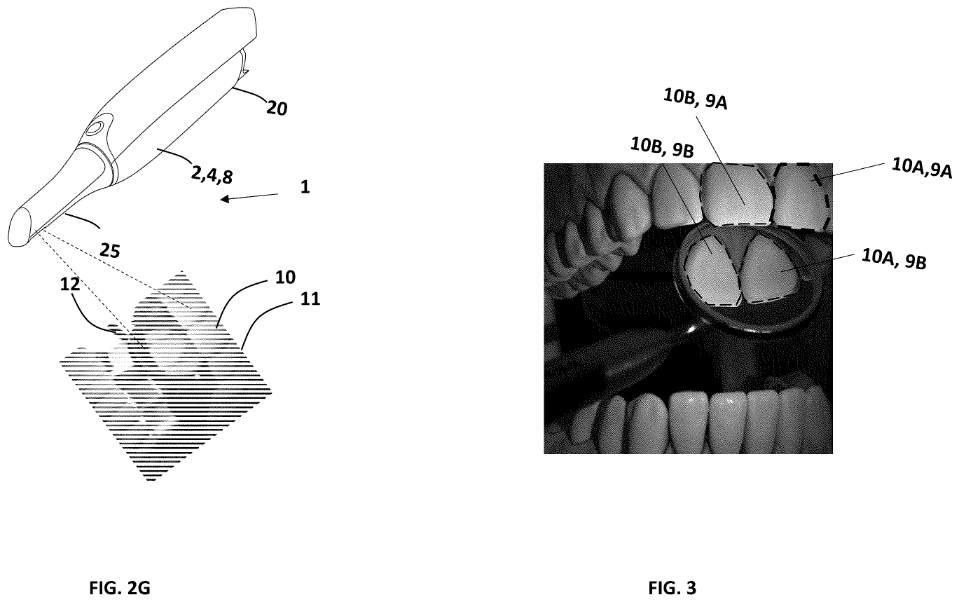

FIGS. 2A to 2G illustrate different examples of the extraoral scanner system 1. In the examples, the system 1 includes a headwear 20 that includes the pattern generator 4, the optical system 8 and the at least one camera 2. The at least one camera may be arranged such that when the user is able to visually see the dental object with at least one eye either directly, i.e. via the first optical path 9A, or via the intraoral mirror device 12, i.e. via the second optical path 9B, then the at least one camera 2 is configured to capture the reflected light of a dental object 10. The reflective surface of the intraoral mirror device 12 should be angled such that the user of the system 1 is able to visually see the dental object 10 of the oral cavity 11. Thereby, the user is able to visually inspect the dental object 10 while determining 3D geometry of the dental object 10. Additionally, while the user is inspecting a 3D model would be generated based on the 3D geometry and displayed on a display. In FIGS. 2A to 2D, the headwear is a pair of glasses. In this example, the at least one camera 2, the pattern generator 4 and the optical system 8 are arranged on the same side of the headwear 20, and the field-of-view of both the pattern generator 4 and the at least one camera 4 are fully overlapping. The at least one camera 2 and the pattern generator 4 are tilted such that an angle is obtained between them. In FIG. 2B, the system 1 includes at least a second camera 2B. In this example, the system 1 includes a first camera and a second camera, and they are both arranged symmetrically around the pattern generator 4 and the optical system 8. In FIG. 2C, the first camera and the second camera are arranged asymmetrically around the pattern generator 4 and the optical system 8. In both FIGS. 2B and 2C, both cameras are tilted such that an angle a is formed between a viewing axis (21A,21B) of both cameras (2A,2B). The angle a should be above 5 degrees for the purpose of utilizing the cameras (2A,2B) for triangulation in determining the 3D geometry. In FIGS. 2B and 2C the patten generator 4 and the optical system 8 in the middle of the headwear such that equal distances are obtained to the first and the second camera (2A,2B). In FIG. 2D, the system 1 includes four cameras (2A,2B,2C,2D), two on each side of the headwear. The two groups of two cameras (2A,2B) and (2C,2D) are symmetrically arranged around the pattern generator 4. In FIG. 2G, the headwear 20 is a headband. In FIG. 2E the headwear is a pair of glasses with an inbuild display in the glasses. In this example, the first and second cameras (2A,2B) are arranged in front of the eyes of the operator for mimicking the field of vision of the operator's eyes. In FIG. 2F, the headwear is a simple headband with the camera 2, the pattern generator 4 and the optical system 8 arranged on the same side of the operator's head.

In FIG. 2G, the headwear 20 is replaced by an intraoral scanner device 20 that includes at least the one camera 2, the pattern generator 4, the optical system 8, and a tip 25 configured to be inserted into the oral cavity 11. In this example, the intraoral scanner device 20 is not being intentional used. In this example, the patient may not be able to open his/hers mouth such that the intraoral scanner device 20 is able to fit into the mouth of the patient. In this example, it would be beneficial to use the intraoral mirror device 12 for the use as being described earlier. The size of intraoral mirror device 12 suits better into the mouth of the patient than the size of the intraoral scanner device 20, and thereby, it would be possible to perform an intraoral scan by the use of the intraoral mirror device 12 even though the patient has difficulties in opening his/hers mouth.

FIG. 3 illustrates an example of where a first dental object 10A and a second dental object 10B is being captured by the at least one camera or multiple cameras of the system 1 via the first optical path 9A and the second optical path 9B via the intraoral mirror device. In this example, the one or more processors 13 is configured to determine a plurality of 2D dental data based on captured 2D images via the first optical path 9A and to determine a plurality of 3D mirror dental data based on captured 2D images via the second optical path 9B. The one or more processors is then further configured to stitch the plurality of 2D dental data together with the plurality of 2D mirror dental data for determining the 3D geometry of at least the first and the second dental object (10A,10B). In this example the 3D geometry of the two dental objects (10A,10B) includes both a facial part and a lingual part.

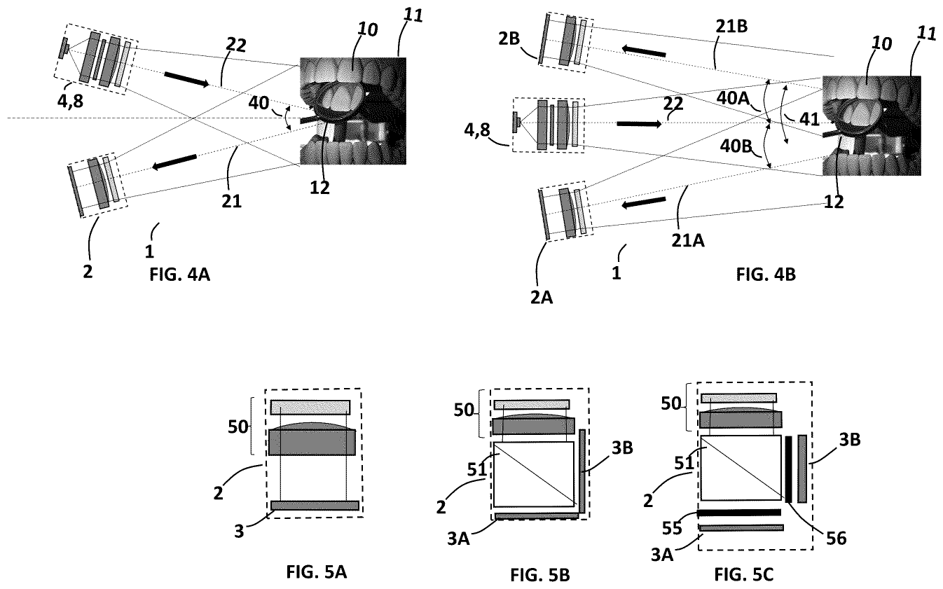

FIGS. 4A and 4B illustrate different configurations of the arrangement including the pattern generator 4, the optical system 8 and the at least one camera 2. In FIG. 4A, the at least one camera 2 has a camera field-of-view axis 21, and the pattern generator 4 with the optical system 8 has a light field-of-view axis 22, and the camera and the pattern generator are arranged such that an angle 40 between the light field-of-view axis 22 and the camera field-of-view axis 21 is between 4° and 200, 6° and 10°, about 8° or about 10°. In FIG. 4A, the system 1 includes two cameras (2A,2B) each having a camera field-of-view axis (21A,21B). The angle (40A,40B) between each of the camera field-of-view axis and the light field-of-view axis 22 are the same or about the same. Both camera field-of-view axes (21A,21B) are overlapping with each other and with the light field-of-view axis (22). An angle 41 between the two-camera field-of-view axes (21A,21B) is formed and is above 6 degrees, above 10 degrees or above 15 degrees.

FIGS. 5A, 5B, and 5C illustrate different examples of the camera 2. In FIG. 5A, the camera 2 includes a single image sensor 3 and with an optical lens system 50 in front to collimate the incident light beam before being captured by the single image sensor 3. In FIG. 5B, the camera 2 includes two image sensors (3A, 3B) and in front of both sensors (3A,3B) a dichroic mirror 50 is arranged. The dichroic mirror 50 is arranged between the optical lens system 50 and the first image sensor 3A and between the optical lens system 50 and the second image sensor 3B. The dichroic mirror 50 is configured to reflect the incident beam within a first group of wavelengths to the second image sensor 3B, and the dichroic mirror 50 is further configured to transmit the incident beam with a second group of wavelengths to the first image sensor 3A.

In FIG. 5Ca narrow bandpass filter 55 is arranged between the first image sensor 3A and the beam splitter 51, and another filter 56 is arranged between the second image sensor 3B and the beam splitter 51, and the another filter 56 is configured to transmit the reflected light from above the wavelength of the probe light. The center wavelength of the narrow bandpass filter should be similar to the wavelength of the probe light. This will reduce the effect of background light in the scanning situation. Ideally, the operator can keep the probe light on during the scanning since this will allow for the most seamless integration into the existing workflow. But if the bright dental light from the chair remains so strong that it reduces contrast needed for 3D scanning, then it may be necessary to turn off the dental light.

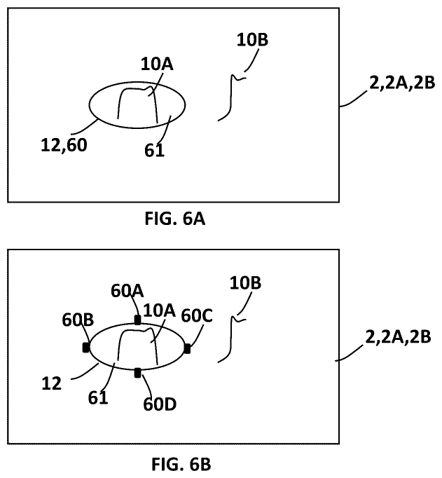

FIGS. 6A and 6B illustrate an example wherein the one or more processors 13 is configured to identify the intraoral mirror device 12 via the at least one camera 2 (or by the cameras (2A,2B)), and wherein the one or more processors 13 is further configured to identify the light returned via the intraoral mirror device based on the identification of the intraoral mirror device. The second optical path is then determined by the one or more processors 13 by detecting via the at least one camera 2 or the cameras (2A, B) one or more identifiers (60,60A,60B,60C,60D) of the intraoral mirror device 12. The one or more processors 13 is configured to identify a mirror surface plane 61 of the intraoral mirror device 12 by tracking the one or more identifiers (60,60A,60B,60C,60D) via the at least one camera 2 or the cameras (2A,2B), and wherein the one or more processors 13 is configured to identify the light returned via the intraoral mirror device 12 based on the identified mirror surface plane 61. In FIG. 6A, the identifier 60 is the geometry of the mirror surface plane 61, and the one or more processors 13 is configured to identify the geometry 60 of the mirror surface plane 61 by using a first neural network, and wherein the one or more processors 13 is configured to identify the light returned via the intraoral mirror device 12 based on the geometry 60 of the mirror surface plane 61. In this example, the first neural network is trained based on a plurality of different geometry of intraoral mirror devices. In FIG. 6B, the intraoral mirror device 12 includes multiple identifiers (60A,60B,60C,60D) which are known markers applied on the intraoral mirror device 12. The one or more processors 13 knows the geometry formed by the identifiers and also the shape of the identifiers (60A,60B,60C,60D), and based on the shape and the geometry the one or more processors 13 is configured to identify the intraoral mirror device 12. The shape and the geometry of the identifiers may be stored in a memory of the system 1. In FIG. 6B, the identifiers (60A,60B,60C,60D) could be small protrusions or markers that are engraved into a frame of the intraoral mirror device 12.

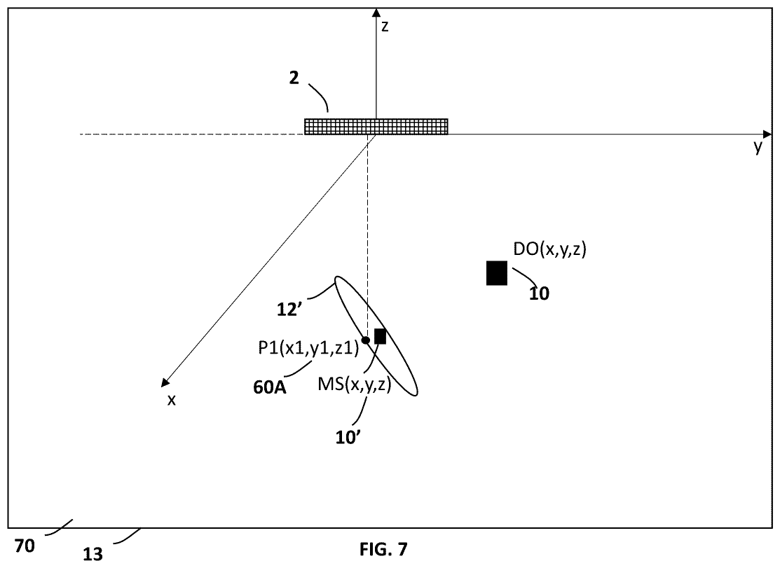

FIG. 7 illustrates an example of the one or more processors configured to transform the captured mirror dental data that are depicted on the intraoral mirror device 12 to an actual position of the mirror dental data on the dental object 10. At least a part of the surface of the dental object 10 is defined at a dental object plane (DO), and the same part of the surface of the dental object 10′ that are also depicted on the intraoral mirror device 12 is defined at a mirror surface plane (MS). The plurality of 2D mirror data includes mirror surface information of the dental object 10′ depicted on the intraoral mirror device 12. The mirror surface information corresponds to 2D properties of a projection of the 3D dental object 10 in the oral cavity 11. The identified intraoral mirror device 12, the camera 2, the dental object plane (DO) and the mirror surface plane (MS) are all arranged in a cartesian coordinate system 70, where in this example, the camera 2 defines the origin of the coordinate system 70. The one or more processors 13 is then configured to transform the mirror surface information from the mirror surface plane (MS) to the dental object plane (DO) by a scaling function. Each of the plurality of 2D dental data includes surface information of the dental object that corresponds to the dental object plane, and each of the plurality of 2D mirror dental data includes mirror surface information of the dental object that corresponds to the mirror surface plane, and when the one or more processors has transformed the mirror surface information from the mirror surface plane to the dental object plane by the scaling function, the one or more processors would stitch the plurality of 2D dental data together with the transformed plurality of 2D mirror dental data to determine the 3D geometry of at least the part of the surface of the dental object 10.

The scaling function is determined by knowing a position (P1) of the intraoral mirror device 12 relative to the camera 2 and based on the position (P1) the one or more processors 13 is configured to determine the scaling function. The one or more processors 13 may determine the scaling function based on the position (P1) by using a mirror surface reconstruction method. The position (P1) may be determined by the one or more processors (P1) by identifying the intraoral mirror device 12 by using the first neural network, and then by a first position algorithm the position (P1) is determined based on an input from the first neural network. As previously described, the first neural network is trained based on a plurality of different geometry of intraoral mirror devices, and the output of the first neural network is a classification of the returned light into whether the returned light is related to the first 9A or the second 9B optical path. Furthermore, the output of the first neural network which serves as an input to the first position algorithm may include dimensions of the identified intraoral mirror device. The first position algorithm is configured to determine the position (P1) by using the known dimensions of the identified intraoral mirror device 12 and trigonometry.

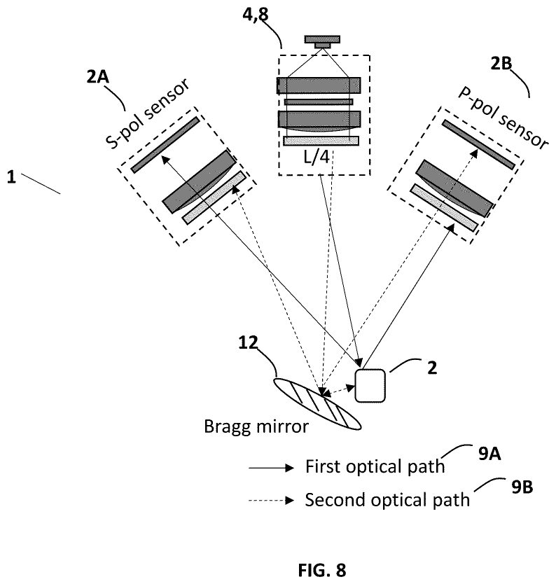

FIG. 8 illustrates yet another example on how to determine whether the reflected light captured by at least one camera 2 is relating to the first optical path 9A or the second optical path 9B. In this example, the intraoral mirror device 12 is a Bragg mirror that is configured to change the polarization of the reflected light that relates to the second optical path 9B. In the example, the polarization of the reflected light in the first optical path 9A is different from the polarization of the reflected light in the second optical path 9B, and the system 1 includes a first camera 2A that is configured to capture reflected light with a first polarization, e.g. S-polarization, and a second camera 2B that is configured to capture reflected light with a second polarization, e.g. P-polarization. Thereby, with less computational power in comparison to the previous example in FIG. 7, the one or more processors 12 would be able to distinguish between reflected light that relates to the first optical path 9A and the second optical path 9B.

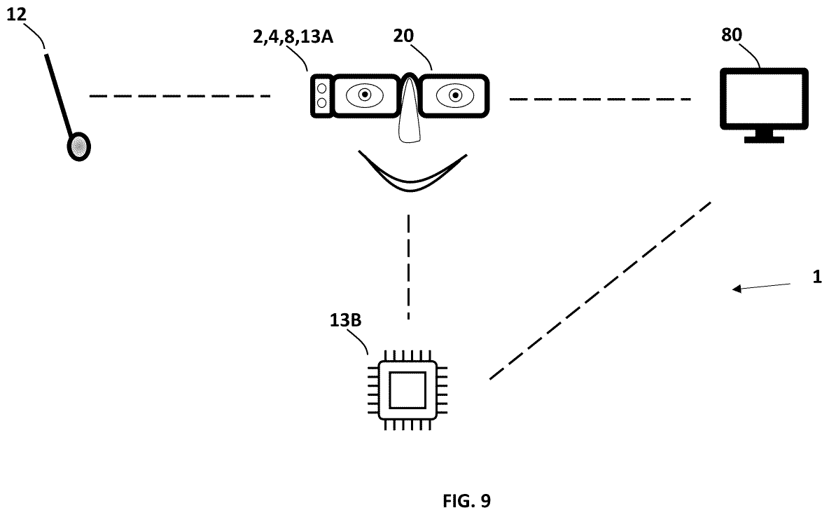

FIG. 9 illustrates the system 1 that includes the intraoral mirror device 12, a headwear 20 that includes the pattern generator 4, the optical system 8 and at least one camera 2, and wherein the headwear 20 is configured to wireless communicate in real time the plurality of 2D dental data and the plurality of 2D mirror dental data or the determined 3D geometry to a display 80 and/or an external processor 13B. In this example, the one or more processors 13 includes two processors (13A,13B), one 13A that is arranged within the headwear 20 and the external processor 13B which is arranged externally to the headwear 20. In this example, the one processor 13A is configured to prepare the plurality of 2D dental data and the 2D mirror dental data to be wireless transmitted to the display 80 and/or the external processor 13B.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

### First List of Items

1. An extraoral scanner system for determining a 3D geometry of at least a part of the surface of a dental object in an oral cavity:
   at least one camera accommodating an array of sensor elements;
   a pattern generator configured to generate, using a light source of the extraoral scanner system, a probe light with a plurality of configurations in the form of an illumination pattern;
   an optical system configured to transmit the probe light towards the dental object along a first optical path of the optical system thereby illuminating at least a part of the dental object with the illumination pattern, and to transmit at least a part of the light returned from the dental object to the at least one camera to form a plurality of 2D dental data,
   an intraoral mirror device configured to reflect the probe light towards the dental object along a second optical path between the intraoral mirror device and the at least one camera, and to transmit at least a part of the light returned from the dental object via the intraoral mirror device to the at least one camera to form a plurality of 2D mirror dental data, and
   one or more processors configured to determine the 3D geometry of at least a part of the surface of the dental object based on the plurality of 2D dental data and the plurality of 2D mirror dental data and the illumination pattern.
2. The extraoral scanner system according to item 1, wherein the one or more processors is configured to identify the intraoral mirror device via the at least one camera, and wherein the one or more processors is further configured to identify the light returned via the intraoral mirror device based on the identification of the intraoral mirror device.
3. The extraoral scanner system according any of the previous items, wherein the second optical path is determined by the one or more processors by detecting via the at least one camera one or more identifiers of the intraoral mirror device.
4. The extraoral scanner system according to item 3, wherein the one or more processors is configured to determine a mirror surface plane of the intraoral mirror device by tracking the one or more identifiers via the at least one camera.
5. The extraoral scanner system according to item 4, wherein the one or more identifiers includes at least a geometry of the intraoral mirror device, and the one or more processors is configured to identify the at least geometry of the intraoral mirror device by using a first neural network.
6. The extraoral scanner system according to item 5, wherein the first neural network is trained based on a plurality of different geometry of intraoral mirror devices.
7. The extraoral scanner system according to item 3, wherein the at least the part of surface of the dental object includes a dental object plane, and each of the plurality of 2D dental data includes surface information of the dental object that corresponds to the dental object plane, and each of the plurality of 2D mirror dental data includes mirror surface information of the dental object that corresponds to the mirror surface plane, and wherein the one or more processors is configured to transform the mirror surface information of each of the plurality of 2D mirror dental data from the mirror surface plane to the dental object plane by a scaling function.
8. The extraoral scanner system according to item 7, wherein the one or more processors is configured to determine the scaling function, and wherein the scaling function is determined based on a position of the mirror surface plane relative to the at least one camera.
9. The extraoral scanner system according to item 7, wherein the one or more processors is configured to determine the scaling function, and wherein the scaling function is determined by a mirror surface angle of the mirror surface plane relative to the at least one camera and a distance between the mirror surface plane and the at least one camera.
10. The extraoral scanner system according to any of the items 7 to 9, wherein the scaling function is determined by the one or more processors by identifying the intraoral mirror device
11. The extraoral scanner system according to any of the previous items, wherein the second optical path is determined in real time.
12. The extraoral scanner system according to any of the previous items, wherein the one or more processors is configured to determine a first point cloud by performing triangulation of each of the plurality of 2D dental data, and wherein the one or more processors is configured to determine a second point cloud by performing triangulation of each of the plurality of 2D mirror dental data, and the one or more processors is configured to determine the 3D geometry by combining the first point cloud and the second point cloud.
13. The extraoral scanner system according to any of the previous items, wherein the illumination pattern is a static pattern that includes-dark and bright regions, the illumination pattern is a time-varying illumination pattern, or the illumination pattern is a pseudo-random pattern.
14. The extraoral scanner system according to any of the previous items, comprising a plurality of high-speed camera that includes the at least one camera, and wherein the plurality of high-speed camera is configured to form the plurality of 2D dental data and the plurality of 2D mirror dental data.
15. The extraoral scanner system according to item 16, wherein the plurality of high-speed camera is arranged symmetrically or asymmetrically according to the light source.
16. The extraoral scanner system according to any of items 14 and 15, wherein each of the plurality of high-speed camera has a camera field-of-view axis, and the pattern generator has a light field-of-view-axis, and the plurality of high speed camera is arranged according to the light source such that an angle for each of the plurality of high-speed camera between the camera field-of-view axis and the light field-of-view-axis is between 4° and 20°, 6° and 10°, about 8° or about 10°.
17. The extraoral scanner system according to any of items 14 to 16, wherein the one or more processors is configured to determine corresponding features across the plurality of 2D dental data and the plurality of 2D mirror dental data by solving a correspondence problem algorithm and perform triangulation of the corresponding features to obtain 3D surface information from the plurality of 2D dental data and the plurality of 2D mirror dental data, and the one or more processors is configured to determine the 3D geometry of the dental object based on the plurality of 3D surface information.
18. The extraoral scanner system according to any of the previous items, comprising a handheld intraoral scanner that includes the at least one camera, the pattern generator, the optical system and a tip configured to be inserted into the oral cavity.
19. The extraoral scanner system according to any of the item 1 to 17, comprising a headwear that includes the at least one camera, the pattern generator, and the optical system.
20. The extraoral scanner system according to any of the previous items, wherein the probe light includes one or more wavelengths between 380 nm and 425 nm.
21. The extraoral scanner system according to any of the previous items, comprising two or more light sources, wherein the two or more light sources includes the light source configured to emit light at a first wavelength and at least another light source configured to emit light at a second wavelength, and wherein the system is configured to switch between the light source and the at least another light source.
22. The extraoral scanner system according to any of the previous items, wherein the system is configured to be in a mode of operation such as, a scan mode for acquiring the intraoral scan data, a loupe mode for acquiring still images or video images at different zoom settings of the at least one camera, a color imaging mode for acquiring color imaging with a higher resolution compared to the scan mode, and/or an infrared imaging mode for acquiring images of within the at least dental object.
23. The extraoral scanner system according to item 18 or 19, wherein the handheld intraoral scanner or the headwear includes a wireless interface configured to transmit the plurality of 2D dental data and the plurality of 2D mirror dental data or the 3D geometry of at least the part of the surface of the dental object.
24. The extraoral scanner system according to item 18 or 19, comprising a power management unit configured to optimize the power consumption of the headwear or the handheld intraoral scanner.
25. The extraoral scanner system according to item 24, wherein the power management unit is configured to set the extraoral scanner system into different power modes based on an input from the at least one camera, wherein a dental object is detected by the power management unit or the processing unit, the power management unit is configured to set the extraoral scanner system into either a scan mode, a loop mode, a colour imaging mode, and/or an infrared imaging mode.
26. The extraoral scanner system according to items 24, wherein the power management unit is configured to set the extraoral scanner system into different power modes based on an input from the at least one camera, wherein a dental object is not detected by the power management unit or the processing unit during a period of time, the power management unit is configured to set the extraoral scanner system into a standby mode.
27. The extraoral scanner system according to item 19, wherein the headwear includes a movement detection unit that includes at least a gyroscope and/or an accelerometer configured to detect a movement of the headwear.
28. The extraoral scanner system according to item 27, wherein the power management unit is configured to set the extraoral scanner system into different power modes based on an input from the movement detection unit, wherein the power management unit is configured to set the extraoral scanner system into a different power mode than a standby mode if a movement of the headwear is detected by the movement detection unit during a period of time.
29. The extraoral scanner system according to item 24, wherein the power management unit is configured to set the extraoral scanner system into different power modes based on an input from a user interface.
30. The extraoral scanner system according to items 19 and 29, wherein the user interface is arranged on the headwear, and the user interface includes a button.
31. The extraoral scanner system according to item 27, comprising a controller that includes an autofocusing function configured to control a position of at least an optical lens of an optical lens system of the at least one camera based on an input from the movement unit.
32. The extraoral scanner system according to item 31, wherein the controller is configured to adjust the focusing of the optical lens system based on an input from the movement unit.
33. The extraoral scanner system according to items 21 or 22 and 29, wherein the controller is configured to adjust a position of a field-of view of the at least one camera based on an input from the movement unit.
34. The extraoral scanner system according to items 19 and 33, wherein the at least one camera is arranged on the headwear via a motorized position unit that is controlled by the controller.
35. The extraoral scanner system according to item 34, wherein the motorized position unit is configured to rotate, tilt and/or translate the at least one camera and/or the another at least one camera.
36. The extraoral scanner system according to claim 19, comprising a displaying unit arranged external to the headwear or part of the headwear, wherein the displaying unit is configured to display one or more of the plurality of 2D dental data and/or one or more of the plurality of 2D mirror dental data.
37. The extraoral scanner system according to item 36, wherein the displaying unit is configured to display solely or a combination of still images, video images at different zoom settings, color imaging, infrared imaging mode.
38. The extraoral scanner system according to claim 19 and any of items 36 and 37, wherein the displaying unit is part of the headwear, the system comprises another displaying unit configured to display the same as the displaying unit.
39. The extraoral scanner system according to any of the previous items, comprising a mouth piece made of a transparent material in relation to the emitted light of the projector unit, wherein the mouth piece is configured to maintain an open mouth that includes the at least dental object.
40. The extraoral scanner system according to any of the previous items, comprising a guiding mean unit configured to determine guiding instructions to a displaying unit or to a speaker unit part of the system or a headwear, wherein the guiding instructions includes guidance on how to position a user's head wearing the headwear in order to acquire optimal intraoral scan data, still images, video images, color images and/or infrared images.
41. The extraoral scanner system according to any of the previous items, wherein the headwear is a pair of glasses, a headband, a neckband, a hat or a cap.
42. The extraoral scanner system according to any of the previous items, wherein the light source is configured to emit light within a range of 400 nm to 2000 nm.

### Second List of Items

1A. An extraoral scanner system configured to determine 3D intraoral scan data, comprising:
   a projector unit comprising at least one light source and a pattern generated optical element, wherein the projector unit is configured to emit a pattern of light onto at least a dental object of a patient;
   an at least one camera configured to capture a plurality of images that depict at least a portion of the projected pattern of light on an intraoral surface of the at least dental object,
   a processor unit configured to process the captured plurality of images into 3D intraoral scan data, and
   a headwear including the projector unit and the at least one camera, and
   wherein an arrangement of the projector unit and the at least one camera allows the extraoral scanner system to capture the plurality of images that depict at least a portion of the projected pattern of light on the intraoral surface of the at least dental object within a mouth of a patient.
1B. An extraoral scanner system configured to determine 3D intraoral scan data, comprising:
   a projector unit comprising at least one light source and a pattern generated optical element, wherein the projector unit is configured to emit a pattern of light onto at least a dental object of a patient;
   an at least one camera configured to capture a plurality of images that depict at least a portion of the projected pattern of light on an intraoral surface of the at least dental object, and wherein an arrangement of the projector unit and the at least one camera allows the extraoral scanner system to capture the plurality of images that depict at least a portion of the projected pattern of light on the intraoral surface of that least dental object within a mouth of a patient, and
   a processor unit configured to process the captured plurality of images into 3D intraoral scan data.
2. The extraoral scanner system according to item 1A or 1B, where the arrangement is part of a headwear.
3. The extraoral scanner system according to item 2, wherein the headwear comprises at least a second camera configured to acquire reflected light from at least the dental object, and wherein the at least one camera and the at least second camera are arranged symmetrically around the projector unit.
4. The extraoral scanner system according to item 2, wherein the headwear comprises at least a second camera configured to acquire reflected light from at least the dental object, and wherein the at least one camera and the at least second camera are arranged asymmetrically around the projector unit.
5. The extraoral scanner system according to any of the previous items, wherein the projector unit includes a projector field of view axis, and the at least one camera includes a first image field of view axis, and the projector field of view axis is not parallel to the first image field of view axis.
6. The extraoral scanner system according to any of items 3 or 4 and item 5, wherein the at least second camera includes a second image field of view axis, and wherein the second image field of view axis is not parallel to the projector field of view axis.
7. The extraoral scanner system according to item 6, wherein a first angle between the first image field of view axis and the projector field of view axis, and a second angle between the second image field of view axis and the projector field of view axis are within a range of 0.1 to 14 degrees, 2 to 10 degrees, or 4 to 9 degrees.
8. The extraoral scanner system according to any of the previous items, wherein the system is configured to be in a mode of operation such as, a scan mode for acquiring the intraoral scan data, a loupe mode for acquiring still images or video images at different zoom settings, a color imaging mode for acquiring color imaging with a higher resolution compared to the scan mode, and/or an infrared imaging mode for acquiring images of within the at least dental object.
9. The extraoral scanner system according to item 8, wherein the system is configured to be in a combined mode including a combination of two or more modes of operation.
10. The extraoral scanner system according to items 1 and/or 3, wherein the at least one camera and/or the at least second camera includes at least a first image sensor and an optical lens system.
11. The extraoral scanner system according to item 10, wherein the at least one camera and/or the at least second camera includes at least a second image sensor.
12. The extraoral scanner system according to item 11, wherein the optical lens system is configured to be shared by the first image sensor and the second image sensor.
13. The extraoral scanner system according to item 12, wherein the at least one camera comprises a dichroic mirror arranged between the optical lens system and the first image sensor and between the optical lens system and the second image sensor.
14. The extraoral scanner system according to any of the previous items, comprising a linearly polarizer unit and a parallel linear filter, and wherein the emitted light is linearly polarized by the linearly polarizer unit, and the parallel linear filter is arranged such that the reflected light is filtered before being captured by the at least one camera.
15. The extraoral scanner system according to any of the previous items, wherein the headwear includes a wireless interface configured to transmit the processed 2D intraoral scan data or the 3D intraoral scan data.
16. The extraoral scanner system according to any of items 1 to 14, comprising an external unit configured to be connected to the headwear, and wherein the external unit includes the processor unit.
17. The extraoral scanner system according to item 16, wherein the connection between the external unit and the headwear is wired or wirelessly.
18. The extraoral scanner system according to any of the previous items, wherein the headwear includes a battery unit that includes a rechargeable battery.
19. The extraoral scanner system according to any of the previous items, comprising a power management unit configured to optimize the power consumption of the headwear.
20. The extraoral scanner system according to item 9 and 19, wherein the power management unit is configured to set the extraoral scanner system or at least the headwear into different power modes based on an input from the at least one camera, wherein a dental object is detected by the power management unit or the processing unit, the power management unit is configured to set the extraoral scanner system or at least the headwear into either the scan mode, the loop mode, the color imaging mode, and/or the infrared imaging mode.
21. The extraoral scanner system according to items 19, wherein the power management unit is configured to set the extraoral scanner system or at least the headwear into different power modes based on an input from the at least one camera, wherein a dental object is not detected by the power management unit or the processing unit during a period of time, the power management unit is configured to set the extraoral scanner system or at least the headwear into a standby mode.
22. The extraoral scanner system according to any of the previous items, wherein the headwear includes a movement detection unit that includes at least a gyroscope and/or an accelerometer configured to detect a movement of the headwear.
23. The extraoral scanner system according to item 22, wherein the power management unit is configured to set the extraoral scanner system or at least the headwear into different power modes based on an input from the movement detection unit, wherein the power management unit is configured to set the extraoral scanner system or at least the headwear into a different power mode than a standby mode if a movement of the headwear is detected by the movement detection unit during a period of time.
24. The extraoral scanner system according to item 19, wherein the power management unit is configured to set the extraoral scanner system or at least the headwear into different power modes based on an input from an user interface.
25. The extraoral scanner system according to item 24, wherein the user interface is arranged on the headwear, and the user interface includes a button.
26. The extraoral scanner system according to items 10 or 11 and 22, comprising a controller that includes an autofocusing function configured to control a position of at least an optical lens of the optical lens system based on an input from the movement unit.
27. The extraoral scanner system according to items 10 or 11 and 22, wherein the controller is configured to adjust the focusing of the optical lens system based on an input from the movement unit.
28. The extraoral scanner system according to items 10 or 11 and 22, wherein the controller is configured to adjust a position of a field-of view of the at least one camera and/or another at least one camera arranged on the headwear based on an input from the movement unit.
29. The extraoral scanner system according to item 28, wherein the at least one camera and/or the another at least one camera is arranged on the headwear via a motorized position unit that is controlled by the controller.
30. The extraoral scanner system according to item 29, wherein the motorized position unit is configured to rotate, tilt and/or translate the at least one camera and/or the another at least one camera.
31. The extraoral scanner system according to any of the previous items, comprising a displaying unit arranged external to the headwear or part of the headwear, wherein the displaying unit is configured to display at least the processed 2D intraoral scan data or the 3D intraoral scan data.
32. The extraoral scanner system according to item 31, wherein the displaying unit is configured to display solely or a combination of still images, video images at different zoom settings, color imaging, infrared imaging mode.
33. The extraoral scanner system according to any of items 31 and 32, wherein the displaying unit is part of the headwear, the system comprises another displaying unit configured to display the same as the displaying unit.
34. The extraoral scanner system according to any of the previous items, comprising a mouth piece made of a transparent material in relation to the emitted light of the projector unit, wherein the mouth piece is configured to maintain an open mouth that includes the at least dental object.
35. The extraoral scanner system according to any of the previous items, comprising a guiding mean unit configured to determine guiding instructions to the displaying or to a speaker unit part of the system or the headwear, wherein the guiding instructions includes guidance on how to position a user's head wearing the headwear in order to acquire optimal intraoral scan data, still images, video images, color images and/or infrared images.
36. The extraoral scanner system according to any of the previous items, wherein the headwear is a pair of glasses, a headband, a neckband, a hat or a cap.
37. The extraoral scanner system according to any of the previous items, wherein the projector unit includes at least one light source configured to emit light within a range of 400 nm to 2000 nm.

1. An extraoral scanner system for determining a 3D geometry of at least a part of the surface of a dental object in an oral cavity:
at least one camera accommodating an array of sensor elements;
a pattern generator configured to generate, using a light source of the extraoral scanner system, a probe light with a plurality of configurations in the form of an illumination pattern;
an optical system configured to transmit the probe light towards the dental object along a first optical path of the optical system thereby illuminating at least a part of the dental object with the illumination pattern, and to transmit at least a part of the light returned from the dental object to the at least one camera to form a plurality of 2D dental data,
an intraoral mirror device configured to reflect the probe light towards the dental object along a second optical path between the intraoral mirror device and the at least one camera, and to transmit at least a part of the light returned from the dental object via the second optical path to the at least one camera to form a plurality of 2D mirror dental data, and
one or more processors configured to determine the 3D geometry of at least a part of the surface of the dental object based on the plurality of 2D dental data and the plurality of 2D mirror dental data and the illumination pattern.

2. The extraoral scanner system according to claim 1, wherein the one or more processors is configured to identify the intraoral mirror device via the at least one camera, and wherein the one or more processors is further configured to identify the light returned via the intraoral mirror device based on the identification of the intraoral mirror device.

3. The extraoral scanner system according to claim 1, wherein the second optical path is determined by the one or more processors by detecting via the at least one camera one or more identifiers of the intraoral mirror device.

4. The extraoral scanner system according to claim 3, wherein the one or more processors is configured to identify a mirror surface plane of the intraoral mirror device by tracking the one or more identifiers via the at least one camera, and wherein the one or more processors is configured to identify the light returned via the intraoral mirror device based on the identified mirror surface plane.

5. The extraoral scanner system according to claim 4, wherein the one or more identifiers includes a geometry of the mirror surface plane, and the one or more processors is configured to identify the geometry of the mirror surface plane by using a first neural network, and wherein the one or more processors is configured to identify the light returned via the intraoral mirror device based on the geometry of the mirror surface plane.

6. The extraoral scanner system according to claim 5, wherein the first neural network is trained based on a plurality of different geometry of intraoral mirror devices.

7. The extraoral scanner system according to claim 3, wherein the at least the part of surface of the dental object includes a dental object plane, and each of the plurality of 2D dental data includes surface information of the dental object that corresponds to the dental object plane, and each of the plurality of 2D mirror dental data includes mirror surface information of the dental object that corresponds to the mirror surface plane, and wherein the one or more processors is configured to transform the mirror surface information from the mirror surface plane to the dental object plane by a scaling function.

8. The extraoral scanner system according to claim 7, wherein the one or more processors is configured to determine the scaling function, and wherein the scaling function is determined based on coordinates of the mirror surface plane relative to the at least one camera.

9. The extraoral scanner system according to claim 7, wherein the one or more processors is configured to determine the scaling function, and wherein the scaling function is determined by a mirror surface angle of the mirror surface plane relative to the at least one camera and a distance between the mirror surface plane and the at least one camera.

10. The extraoral scanner system according to claim 7, wherein the scaling function is determined by the one or more processors by identifying the intraoral mirror device

11. The extraoral scanner system according to claim 1, wherein the second optical path is determined in real time.

12. The extraoral scanner system according to claim 1, wherein the one or more processors is configured to determine a first point cloud by performing triangulation of each of the plurality of 2D dental data, and wherein the one or more processors is configured to determine a second point cloud by performing triangulation of each of the plurality of 2D mirror dental data, and the one or more processors is configured to determine the 3D geometry by combining the first point cloud and the second point cloud.

13. The extraoral scanner system according to claim 1, wherein the illumination pattern is a static pattern that includes-dark and bright regions, or, the illumination pattern is a time-varying illumination pattern, or, the illumination pattern is a pseudo-random pattern.

14. The extraoral scanner system according to claim 1, comprising a plurality of high-speed camera that includes the at least one camera, and wherein the plurality of high-speed camera is configured to generate the plurality of 2D dental data and the plurality of 2D mirror dental data.

15. The extraoral scanner system according to claim 14, wherein the plurality of high-speed camera is arranged symmetrically or asymmetrically according to the light source.
